(19) United States
(12) Patent Application Publication
(10) Pub. No.: US 2020/0262991 A1
(43) Pub. Date: Aug. 20, 2020

(54) Method for Tuning Topology of Polymer Particles

(71) Applicant: UNIVERSITY OF SOUTH CAROLINA, COLUMBIA, SC (US)

(72) Inventors: R. MICHAEL GOWER, Columbia, SC (US); CHRISTOPHER ISELY, COTTAGE GROVE, MN (US)

(21) Appl. No.: 16/707,449

(22) Filed: Dec. 9, 2019

Related U.S. Application Data
(60) Provisional application No. 62/805,458, filed on Feb. 14, 2019.
(60) Provisional application No. 62/833,946, filed on Apr. 15, 2019.

(51) Int. Cl. C08J 7/02; C08J 3/09; C08J 3/14; G01N 33/58; A61K 31/05; A61K 9/16; A61K 31/4439

(52) U.S. Cl. CPC C08J 7/02; C08J 3/093; C08J 3/14; G01N 33/582; C08J 2367/04; A61K 9/1694; A61K 9/1647; A61K 31/4439; A61K 31/05

(57) ABSTRACT

Methods for forming polymeric particles with a predetermined, controlled morphology, and methods for tuning the topology of polymeric particles through modification of components utilized in the formation process are described. An emulsion/condensation technique is used, including a first emulsifier in the aqueous phase and an organic phase that includes first and second solvents, a second emulsifier, and a polymer. The polymer is more soluble in the first solvent than the second solvent, and the second emulsifier is more soluble in the second solvent than the first solvent. Through modification of the relative amounts of the emulsifiers and solvents, particles with either smooth or rough surfaces can be formed. Particles are particularly useful as drug depots with controlled-release profiles.

### CROSS REFERENCE TO RELATED APPLICATION

This application claims filing benefit of U.S. Provisional Patent Application Ser. No. 62/805,458, entitled “Development of Microparticles for Controlled Release of Resveratrol to Adipose Tissue and the Impact of Drug Loading on Particle Morphology and Drug Release,” having a filing date of Feb. 14, 2019; and of U.S. Provisional Patent Application Ser. No. 62/833,946, entitled “Method for Tuning Topology of Polymer Particles,” having a filing date of Apr. 15, 2019, both of which are incorporated herein by reference for all purposes.

### FEDERAL RESEARCH STATEMENT

This invention was made with government support under Grant No. P20GM103641, awarded by the National Institutes of Health. The Government has certain rights in the invention.

### BACKGROUND

The formation of micro- and nano-sized polyester-based particles has found use in a plurality of different applications. For instance, polymer particles based on biocompatible and biodegradable polyesters have been developed for use as drug depots in controlled release applications. Such particles are generally formed according to an emulsion/condensation approach in which a two-phase emulsion is formed with the polymer contained in the dispersed phase. As solvent is removed from the dispersed phase, the polymer solidifies, forming polymeric particles suspended in the aqueous phase. In the case of drug delivery particles, the drug to be delivered is included in the dispersed phase and upon solidification, the drug is contained by the solidified polymeric matrix. During use, the drug is released as the polymer degrades.

While emulsion/condensation formation approaches can provide small particles in a relatively consistent fashion, the ability to control the surface characteristics of the particles, for instance to provide consistently spherical particles or particles with a higher surface area, has not been as successful. Such issues are particularly evident when forming particles that carry materials in conjunction with the matrix polymer (e.g., a drug to be delivered by the particles), as the addition of materials to the formation process can have unintended consequences on the particles formed by the process.

What are needed in the art are methods for forming polymeric particles that can provide polymeric particles having a predetermined and controlled topology. For instance, methods of forming particles that can consistently provide spherical particles, high surface area particles or a predetermined mixture of both would be of great benefit.

### SUMMARY

Disclosed is a method for forming a polymeric particle according to an emulsion/condensation methodology. A method can include combining an aqueous phase with an organic phase to form an emulsion that includes droplets of the organic phase dispersed in the aqueous phase. The aqueous phase can include water and a first emulsifier. The organic phase can include a first solvent, a second solvent, a polymer, and a second emulsifier. The polymer can include a polyester homopolymer or copolymer. The second emulsifier can include a small molecule amphiphilic compound. The method can also include removing at least a portion of the first solvent from the organic phase, upon which the polymer can solidify to form polymeric particles dispersed in the aqueous phase. In addition, the method can include modifying the concentration of the second emulsifier in the organic phase, and optionally, can also include modifying the concentration of the first emulsifier in the aqueous phase, thereby modifying the topology of the polymeric particles. For instance, by modifying the concentration of the second emulsifier to be about 5 mg/mL or greater in the organic phase, at least a portion of the polymeric particles can exhibit a high surface area topology, e.g., a rough or ruffled topology.

The components of the formation system can have characteristics that can help to tune the morphology and/or the topology of the particles. For instance, at the conditions of formation, the second solvent can be more miscible in the aqueous phase as compared to the first solvent. The second emulsifier can be more soluble in the second solvent as compared to its solubility in the first solvent and the reverse can be true for the polymer, i.e., the polymer can be more soluble in the first solvent as compared to its solubility in the second solvent.

Also disclosed are methods for forming a biodegradable drug delivery particle according to an emulsion/condensation methodology. For instance, a method can include combining an aqueous phase with an organic phase to form an emulsion that includes droplets of the organic phase dispersed in the aqueous phase. The aqueous phase can include water and a first emulsifier. The organic phase can include a first solvent, a second solvent, a polymer, and a second emulsifier. The second emulsifier can exhibit a biological activity or optionally, the organic phase can also incorporate a biologically active agent. The polymer can include a biodegradable and biocompatible polyester homo- or copolymer. The second emulsifier can include a small molecule amphiphilic compound. The method can also include removing at least a portion of the first solvent from the organic phase, upon which the polymer can solidify to form polymeric particles that contain the biologically active agent dispersed in the aqueous phase. The method can further include modifying the concentration of the second emulsifier in the organic phase, and optionally, can also include modifying the concentration of the first emulsifier in the aqueous phase, thereby determining the morphology of the polymeric particles. For instance, by modifying the concentration of the second emulsifier to be about 5 mg/mL or greater in the organic phase, at least a portion of the polymeric particles can exhibit a high surface area topology.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates the effect of ethanol on particle mass yield and size. (A) Vials containing 1:1, 1:2, and 1:3 volume ratios of ethanol and dichloromethane (DCM) with 6% poly(lactide-co-glycolide) (PLG). (B) Mass yield of particles made with an organic phase consisting of 1:1, 1:2, 1:3 volume ratios of ethanol and DCM and DCM only. Data is the mean of three independent particle fabrications and analyzed with one-way ANOVA followed by Tukey's multiple comparisons test. ****p<0.0001. (C, D) Light microscopy images and (E, F) size distribution of particles made with 1:2 and 1:3 mixtures of ethanol and DCM. Scale bar indicates 40 μm.

FIG. 2 illustrates the morphology of resveratrol loaded PLG particles. Representative images of particles made with an oil phase consisting of (A) a 1:3 mixture of ethanol and DCM and 10 mg/mL of resveratrol, (B) a 1:3 mixture of ethanol and DCM and no resveratrol, and (C) DCM with no resveratrol or ethanol. All particles were made with an oil phase that was 6% PLG. The aqueous phase of the emulsion contained 1% polyvinyl alcohol. Scale bars indicate 50 μm.

FIG. 3 illustrates the effect of resveratrol loaded particles on adipocyte lipid content. (A-F) Images of cell monolayers stained with Oil Red O. Experimental groups include (A) undifferentiated fibroblasts, (B) adipocytes with no treatment, (C) adipocytes treated with 300 μg/mL of PLG particles, (D) adipocytes treated with 300 μg/mL of resveratrol particles (E) adipocytes treated with 60 μM resveratrol dissolved in ethanol and suspended in DMEM at 0.05 v/v %, and (F) adipocytes treated with an equivalent amount of ethanol. (G) Image of Oil Red O extracted in isopropanol from the six experimental groups. (H) Absorbance values of Oil Red O extracted from cells using isopropanol at 500 nm. Absorbance correlates to lipid content inside the cells. Labels for (H) correspond to (C-E) in terms of treatment group. The experiment was repeated 4 times. One-way ANOVA was conducted followed by a Tukey multiple comparison test. * p<0.05. Scale bars indicate 500 μm.

FIG. 4 presents in vitro release profiles of resveratrol particles. (A) Six-week resveratrol release profile. Data are mean±standard deviation from one experiment with three replicates per time point. (B) Two-week resveratrol release profile. Data is plotted as mean±standard deviation from two independent experiments with three replicates per time point.

FIG. 5 illustrates the effect of resveratrol concentration on particle morphology. (A-D) Images of resveratrol loaded particles made with decreasing concentrations of resveratrol in the oil phase (shown in parentheses), which was achieved by decreasing the volume ratio of ethanol to DCM. 6% PLG was used in the emulsion. The PVA concentration was 2% in the emulsion step and 1% in the evaporation step. Scale bars indicate 50 μm.

FIG. 6 illustrates the effect of PVA concentration on particle morphology. (A-D) Images of resveratrol particles made with an oil phase consisting of a 1:9 mixture of ethanol to DCM and containing 4 mg/mL resveratrol and 6% PLG. PVA concentrations in the emulsion step and solvent evaporation step are indicated above the image as emulsion %, evaporation %. Resveratrol loading is listed in parentheses. Scale bars indicate 50 μm.

FIG. 7 provides confocal imaging of particles made with FITC-PVA. Fluorescent images of particles made with oil phases consisting of 6% PLG in (A) DCM (B) 1:9 ethanol:DCM, (C) 1:9 ethanol:DCM containing 4 mg/mL resveratrol, and (D) 1:3 ethanol:DCM containing 10 mg/mL resveratrol. FITC signal intensity was quantified using ImageJ and is depicted in panel (E) as mean±standard deviation for 27 particles per group. Scale bar is 20 μm.

FIG. 8 illustrates flow cytometry of resveratrol particles made with FITC-PVA. (A) Histograms depicting FITC fluorescence intensity of particles made with oil phases consisting of 6% PLG in DCM (DCM), 1:9 ethanol:DCM (1:9), and 1:9 ethanol:DCM containing 4 mg/mL resveratrol (1:9 RSV). (B) Mean fluorescence intensity (MFI) and standard deviation of data from panel (A). 600 particles were analyzed for each group.

FIG. 9 illustrates the effect of cosolvent (methanol, acetone, or ethanol) on particle morphology. Light microscopy (A-C) and SEM (D-F) of particles made with an oil phase containing 6% (w/w) PLG and 10 mg/mL of resveratrol. The aqueous phase was 1% (w/v) PVA. The solvent composition of the oil phase was: (A,D) 25% methanol and 75% DCM, (B,E) 25% acetone and 75% DCM, and (C,F) 25% ethanol and 75% DCM. Scale bars indicate 50 μm.

FIG. 10 illustrates the impact of resveratrol on the morphology of polycaprolactone (PCL) and polylactide (PLA) particles. Light microscopy of particles made with 6% (w/w) of PCL (A,B) or 6% (w/w) PLA (C,D). In addition, the oil phase consisted of 25% ethanol and 75% dichloromethane and contained resveratrol at either 10 mg/mL (A,C) or 0 mg/mL (B,D). The aqueous phase consisted of 1% (w/v) polyvinyl alcohol. Scale bars indicate 50 μpm.

FIG. 11 illustrates light microscopy images of particles loaded with rosiglitazone and particles loaded with resveratrol.

FIG. 12 illustrates the impact of ethanol and resveratrol on particle morphology. Light microscopy (A-D) and SEM (E-H) images of particles produced from an oil-in-water emulsion. The aqueous phase was 1% (w/v) PVA. The oil phase consisted of 6% (w/w) PLG, resveratrol, DCM, and ethanol. The volume percent of ethanol and the concentration of resveratrol (RSV) are indicated directly above the corresponding light microscopy image.

FIG. 13 illustrates the effect of various trans-stilbenes on particle morphology. Images of particles made with an organic phase of 6% (w/w) PLG, 25% ethanol, 75% dichloromethane, and 10 mg/mL of Trans-Stilbene (A), Pinosylvin (B), Resveratrol (C), or Piceatannol (D). The aqueous phase was 1% (w/v) PVA. Scale bar was 50 um.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, disclosed herein are methods for forming polymeric particles with a predetermined, controlled morphology and/or topology and methods for tuning the morphology and/or topology of polymeric particles through modification of components utilized in the formation process. As utilized herein, the term “topology” generally refers to the presence or lack of surface deformation of a particle, e.g., the existence of features across the surface that lead to additional surface area such as ridges, bumps, dips, etc. or alternatively, a smooth surface. As utilized herein, the term “morphology” generally refers to the overall shape of a particle, e.g., large, small, spherical, ovoid, cubic, etc.

For instance, through modification of the amount or concentration of components utilized in the formation process, particles can be formed having a high surface area topology (i.e., a “rough” or “ruffled” topology) or a smooth surface.

Moreover, a particulate can be formed in which essentially all particles of the particulate have the same topology, e.g., either a rough surface or a smooth surface; or alternatively, a particulate can include a mixture of particles with a predetermined ratio of different topologies and/or morphologies.

Through modification and control of the topology of polymer particles a variety of different particle characteristics can be controlled. For instance, in one embodiment, disclosed particles can be designed for use as drug depots and utilized in delivery of a biologically active agent (e.g., a drug) to a delivery site (e.g., a targeted or systemic in vivo delivery of a drug). Through control and modification of the morphology and topology of polymeric particles carrying the agent, release characteristics as well as cell and tissue interactions can be modified and controlled. For instance, a higher surface area particle can have a higher loading of a biologically active agent as compared to a particle having the same average diameter or volume but having a smooth surface. This difference can be particularly large when the biologically active agent is carried at the particle surface. Moreover, interactivity of a particle with particular tissues or cells can vary depending upon the topology of the particle, and as such, improved delivery to a particular location or particular structures can be attained through control and modification of the particle topography. For instance, particular cells or tissues can exhibit higher interactivity with a particle having a rough surface as compared to a smooth surface. In such an embodiment, formation of a drug delivery depot in the form of particles with a rough surface can increase interaction between the particles and the desired delivery site, which can provide increased drug delivery to the desired site and less off-target delivery. In other embodiments, it may be preferred to form particles having a smooth surface, e.g., to minimize interfacial free energy. Control of the morphology of the particles in combination with control of the topology of the particles can provide additional benefits with regard to loading levels, interaction characteristics, etc.

Particles formed according to disclosed methods are not limited to use in biological applications as drug delivery particles. Improvements as can be attained through the controlled formation techniques disclosed herein are applicable to a wide variety of applications including, without limitation, textiles, coatings applications, filler applications, etc. as particles having well-defined and controlled shapes and add-on levels of carried agents can provide improved hydrophobicity, oleophobicity, rheology, thixotropy, adhesion, etc.

Disclosed particle formation methods are based on oil-in-water emulsion techniques in which an organic phase is combined with an aqueous phase to form an emulsion including droplets of the organic phase dispersed in the aqueous phase. In a typical oil-in-water emulsion technique, the organic phase (also referred to herein as the oil phase) includes a polymer dissolved in an organic solvent. The aqueous phase includes water and an emulsifier that partitions itself at the oil/water interface of the dispersed droplets of the organic phase, thereby maintaining the particle droplets throughout emulsification and solidification and preventing agglomeration of the droplets as well as the solidified particles. Following emulsion formation, solvent is removed from the organic droplets upon which the polymer carried in the organic phase solidifies to form the polymeric particles suspended in the aqueous phase.

Disclosed methods differ from typical oil-in-water emulsion techniques through the addition of a second emulsifier and a second solvent, both being incorporated in the oil phase. These additions provide a system that allows for modulation of the surface roughness, as well as other characteristics (e.g., add-in level of additives, particle size) of the solidified polymer particles. Without wishing to be bound to any particular theory, it is believed that the second solvent can preferentially carry the second emulsifier to the water/oil interface during solidification, leading to the accumulation of the second emulsifier at the interface where the first and second emulsifiers compete with one another. A localized decrease in surface tension at the interface due to the presence of the less effective emulsifier (which is generally the second emulsifier) causes the droplet to expand irregularly across the interface, which, in turn, leads to folding and deforming of the interface that does not reverse prior to polymer hardening. At high enough concentration of the less effective emulsifier, this can result in a polymer particle with a non-smooth, rough or ruffled surface.

Through control of the relative concentrations of the first and second emulsifiers at the interface, the topology of the particles being formed can thus be controlled. For instance, through modification of the concentration of the second solvent and/or the second emulsifier in the oil phase or through modification of how quickly each solvent leaves the oil droplets (e.g., by changing the volume of a stirred water bath used during solidification) the particle surface can be tuned from smooth to slightly ruffled to highly ruffled. Moreover, through inclusion of the second solvent in the organic phase, the loading level of an agent that is highly soluble in the second solvent (as compared to the first solvent) can be increased in the formed particles. This is particularly beneficial when the second emulsifier that is more highly soluble in the second solvent also provides a desired function to the formed particles, e.g., a biological activity in the case of a drug delivery particle.

As stated, the formation of the emulsion is carried out similar to previously known typical emulsion/condensation particle formation processes; however, the oil droplets include both a first and second solvent as well as the polymer and a second emulsifier.

The aqueous phase includes an emulsifier that is soluble in water. This aqueous phase emulsifier encompasses any emulsifier as is generally known in typical emulsion/condensation polyester particle formation techniques. For instance, and without limitation, polyvinyl alcohol, hydroxyethyl cellulose, carboxymethyl cellulose, methyl cellulose, gelatin, alkylarylsulfonates, alkylsulphates, fatty acid salts of alkali metals, polyethylene glycol (PEG), poly(ethylene-alt-maleic acid) (PEMA), didodecyldimethylammonium bromide (DMAB), and mixtures thereof. For instance, a polyvinyl alcohol or other biocompatible emulsifier can be utilized as an emulsifier in some embodiments, and in particular, in those embodiments in which the particles are intended for use in a biological application. In general, the aqueous phase can include an emulsifier in an amount of from about 0.1 w/v % to about 5 w/v %, for instance from about 0.5 w/v % to about 3 w/v % or from about 1 w/v % to about 2 w/v % by volume of the aqueous phase.

There are no particular limitations on the size or type of a polymeric emulsifier that can be utilized. For instance, a polyvinyl alcohol emulsifier can have about 85% or greater hydrolysis of the acetate groups, or about 95% or greater hydrolysis of the acetate groups, or can be fully hydrolyzed in some embodiments. In addition, the size of a polymeric emulsifier is not limited. For instance, the weight average molecular weight of a polymeric emulsifier may be about 5,000 or greater; for instance, from about 5,000 to about 500,000, from about 10,000 to about 200,000, or from about 12,000 to about 50,000 in some embodiments.

The organic phase includes a second emulsifier that is understood to compete with the first emulsifier (that of the aqueous phase) at the oil/water interface during emulsification and solidification. Accordingly, the second emulsifier can be an amphiphilic compound that exhibits surface activity in the emulsion/condensation system. However, the surface tension at a water/oil interface in the presence of the second emulsifier can differ from the surface tension at a similar oil/water interface in the presence of the first emulsifier. As such, local variations in surface tension can be established across the surface of the dispersed oil droplets in disclosed systems, which is believed to lead to the ability to establish topological variations and ‘roughening’ of the surface of solidified particles as described herein.

In addition to the above, the second emulsifier can also exhibit a higher solubility in one of the solvents of the organic phase as compared to a second solvent of the organic phase. This can provide for preferential transport of the second emulsifier to the water/oil interface of dispersed droplets where topological modification can occur.

Second emulsifiers can encompass small molecule amphiphilic compounds. For instance, second emulsifiers can have a number average molecular weight of about 900 g/mol or less, about 500 g/mol or less, or about 300 g/mol or less in some embodiments. For example, a second emulsifier can have a number average molecular weight of from about 100 g/mol to about 400 g/mol, or from about 200 g/mol to about 300 g/mol in some embodiments.

In one embodiment, a second emulsifier can be a small molecule amphiphilic phenolic compound that includes one or more phenolic groups and is amphiphilic at the emulsion formation conditions. In some embodiments, a second emulsifier can be a polyphenolic compound comprising at least two phenol groups joined by a C1-C6 alkyl chain that can optionally include additional functionality (e,g., additional hydroxyl groups, oxygen, unsaturation, etc.).

In some embodiments, the second emulsifier can provide additional functionality to the formed particles. For instance, in some embodiments, a polyphenolic compound that exhibits desirable biological activity can be utilized as the second emulsifier and as such, the particles can be utilized as drug depots for delivery of the second emulsifier from the formed system.

Polyphenolic compounds as may function as both emulsifier and as a biologically active agent that can be delivered from the formed particles can include in one embodiment those described by the following structure:

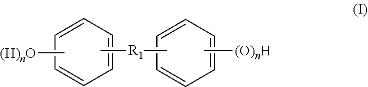

in which R₁ is alkenyl, C(O)CH═CH, or a hydroxy pyranone fused to one of the phenyl moieties to form a flavone; and each n is independently 0-3.

For example, a second emulsifier can be a polyhydroxy stilbene (e.g., polyhydroxy-trans-stilbene) as shown in formula (II), a polyhydroxy chalcone as shown in formula (III), or a polyhydroxyflavone as shown in formula (IV). In one embodiment, the emulsifier can be substituted with at least 2, e.g., 3, 4, or 5 hydroxy moieties.

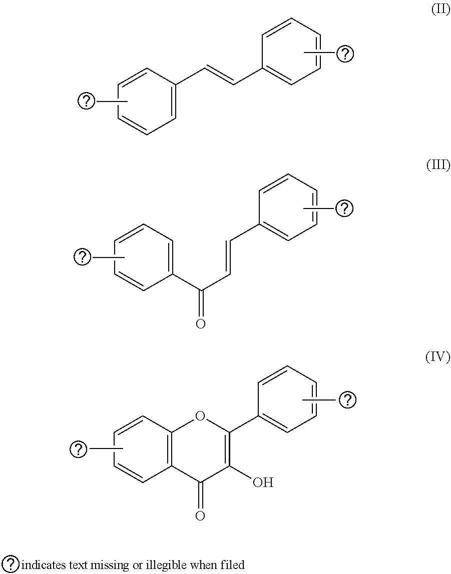

Exemplary compounds can include, without limitation, trans-stilbene, resveratrol (3,5,4′-trihydroxy-trans-stilbene), butein (3,4,2′, 4′-tetrahydroxychalcone), piceatannol (3,5,3′, 4″-tetrahydroxy-trans-stilbene), isoliquiritigenin (4,2′, 4′-trihydroxychalcone), fisetin (3,7,3′, 4′-tetrahydroxyflavone), quercetin (3,5,7,3′, 4′-pentahydroxyflavone), pinosylvin (trans-3,5-dihydroxystilbene), and trans-4-hydroxystilbene.

Resveratrol can be utilized as an emulsifier in one particular embodiment. Resveratrol is a polyphenolic compound occurring in various plants, including grapes, berries, and peanuts, as a defense mechanism against environmental stresses such as infections and ultraviolet radiation. A remarkable range of biological functions are attributed to resveratrol including cancer prevention, anti-inflammatory, antioxidant activities, and as an anti-obesity compound. It exerts the anti-obesity effect by activating signaling pathways in fat cells that are also activated by energy restriction (i.e., dieting). The result is an increase in lipolysis and fatty acid oxidation that leads to a reduction in body fat. Unfortunately, resveratrol suffers from poor bioavailability, requiring large and frequent doses, thereby hampering its vast therapeutic potential. Through utilization of disclosed methods, drug depot particles that incorporate resveratrol at high concentrations can be formed. Moreover, through control of the surface morphology of the particles, improved delivery of the materials can be attained, with less off-target delivery; for instance, as drug depots for controlled release of resveratrol within fat tissues.

The content of the second emulsifier can be modified to control the surface morphology of the particles. For instance, at relatively low concentration, e.g., about 4 mg/mL or less of the oil phase, the second emulsifier can be incorporated in the formed particles and the particles can have a smooth surface. This embodiment may be preferred when the second emulsifier has another function in the particles, e.g., for delivery in a final application, for modification of particle characteristics or of a composition that incorporates the particles, etc. As the concentration of the second emulsifier is increased in the oil phase, the particles can begin to exhibit a rough surface. For instance, at about 5 mg/mL oil phase or higher, such as from about 5 mg/mL to about 10 mg/mL, the particles can exhibit a rough surface.

The concentration of the first emulsifier of the aqueous phase can also be utilized to effect surface morphology of the particles in conjunction with the concentration of the second emulsifier of the oil phase. For instance, when the concentration of the second emulsifier is relatively low, e.g., from about 4 mg/mL of the oil phase to about 6 mg/mL of the oil phase, decreasing the concentration of the first emulsifier in one or both of the emulsification solution and the solidification solution (e.g., to about 2 w/v% or less or about 1 w/v% or less of the aqueous phase) can increase the surface roughness of individual particles and can increase the number of particles that exhibit the surface roughness. At a relatively high concentration of second emulsifier (e.g., about 10 mg/mL oil phase or higher) and a relatively low concentration of first emulsifier (e.g., about 1 w/v% or less of the aqueous phase), essentially all of the formed particles can exhibit a rough surface. Similarly, smooth particles can be formed at relatively high concentration of second emulsifier (e.g., up to about 5 mg/mL oil phase, such as 4 mg/mL) through increase in the concentration of the first emulsifier (e.g., greater than about 2 w/v% of the aqueous phase).

In conjunction with the second emulsifier, the organic phase can include a dissolved polymer, a first solvent, and a second solvent. The polymer of the organic phase can encompass any polyester homo- or copolymer as is generally known in typical emulsion condensation particle formation processes. Polyesters can be amorphous, crystalline, and/or a combination thereof. For instance, any polyester including branched or unbranched, and optionally, including chemical unsaturation that is soluble in a water-immiscible organic solvent is encompassed. A polyester can have any glass transition temperature as long as it is soluble in the first solvent at the formation conditions and can have any weight average molecular weight. In some embodiments, the weight average molecular weight can be from about 5,000 to about 200,000, for instance about 135,000 in some embodiments.

Polyesters can be condensation products of unsaturated polybasic acids or of corresponding acid equivalent derivatives thereof including esters, anhydrides or acid chlorides and polyhydric alcohols. Optionally, one or more additional polyacids common in the art of polycondensation may be used in addition to the unsaturated polyacid. Examples of ethylenically unsaturated polyacids include, but are not limited to maleic, fumaric, itaconic, phenylenediacrylic, citraconic and mesaconic acid. Diacids as may be incorporated in a polyester can include, but are not limited to, malonic, succinic, glutaric, adipic, pimelic, azelaic, and sebacic acids, phthalic, isophthalic, terephthalic, tetrachlorophthalic, tetrahydrophthalic, trimellitic, trimesic, isomers of naphthalenedicarboxylic acid, chlorendic acid, trimellitic acid, trimesic acid, and pyromellitic acid. Polyesters can incorporate any of a wide variety of polyhydric alcohols, which are well known in the art of polycondensation and may be aliphatic, alicyclic, or aralkyl. Exemplary alcohols can include, but are not limited to ethylene glycol, 1,3-propylene glycol, 1,6-hexanediol, 1,10 decanediol, cyclohexanedimethanol, 1,4-cyclohexanediol, hydroquinone bis (hydroxyethyl) ether, diethylene glycol, neopentyl glycol, bisphenols such as bisphenol A, ethylene oxide and propylene oxide adducts of bisphenol A, pentaerythritol, trimethylolpropane, and polyester polyols, such as that obtained by the ring-opening polymerization of ϵ-caprolactone. Additionally, A-B type polycondensation monomers, which contain both hydroxyl and acid derivative functions, can be used, as well as monoacids and monoalcohols.

In some embodiments, particles can be intended for use in biological systems and as such the polyesters used in forming the particles can be biocompatible and, in some embodiments, biodegradable. Biocompatible and biodegradable polyesters as may be utilized can include, without limitation, poly(ethylene oxide)-poly(propylene oxide)-poly(ethylene oxide) (PEO-PPO-PEO) copolymer, poly(lactide-co-glycolide) (PLG) copolymer, polycaprolactone (PCL), poly(lactic acid) (PLA), poly(glycolic acid) (PGA), polyethylene glycol, and polysorbate, as well as any combination of biocompatible, biodegradable polymers.

In general, the organic phase can incorporate the polymeric component in an amount of from about 1 w/w % to about 12 w/w %.

As first solvent, an organic solvent that is soluble for the polyester and slightly soluble or insoluble in the aqueous phase and also non-reactive with other components of the system can be utilized. First solvents can be aliphatic, aromatic, aromatic-aliphatic, saturated or unsaturated, halogenated solvents, an ether, or combinations thereof. Examples of suitable first solvents can include, without limitation, toluene, xylene, dichloromethane, chloroform, trichloroethylene, tetrachloroethylene, tetrachloroethanes, chlorobenzene, dichlorobenzenes, ethyl acetate, butyl acetate, ethyl formate, methylethyl ketone, and mixtures of these compounds. In some embodiments, it may be preferred to utilize as first solvent an organic solvent with relatively low toxicity that has been deemed non-hazardous for use in formation of biocompatible systems. For instance, 1,4-dioxane, dichloromethane, chloroform, dimethylformamide, or dimethylacetamide or mixtures thereof can be utilized as a first solvent in an application intended for biological uses.

The second solvent of the organic phase can be a solvent that is miscible in both water and the first solvent. In addition, the second solvent can solubilize the second emulsifier and can solubilize little or none of the polymer. As such, the second emulsifier can be more soluble in the second solvent as compared to its solubility in the first solvent. In addition, the polymer can be more soluble in the first solvent as compared to its solubility in the second solvent. For instance, the solubility of the second emulsifier in the second solvent at the emulsion formation conditions can be about 20 mg/mL or greater, for instance from about 40 mg/mL to about 500 mg/mL, or even higher in some embodiments, and the solubility of the second emulsifier in the first solvent can be lower than that in the second solvent, for instance about 20 mg/mL or less, for instance from about 2 mg/mL to about 10 mg/mL, or essentially insoluble in some embodiments. The solubility of the polymer in the second solvent at the formation conditions can be low, about 20 mg/mL or less, for instance from about 2 mg/mL to about 10 mg/mL or essentially insoluble in some embodiments and the solubility of the polymer in the first solvent can be higher, for instance about 20 mg/mL or greater, for instance from about 40 mg/mL to about 500 mg/mL, or even higher in some embodiments. However, both the polymer and the second emulsifier must be soluble in a mixture of the first and second solvents.

Examples of exemplary second solvents can include, without limitation, low molecular weight alcohols including methanol, ethanol, and propanol, as well as acetone, tetrahydrofuran, etc., as well as mixtures thereof.

The amount of the second solvent included in the oil phase can vary, depending, for example, on the concentration of second emulsifier to be included in the system and in the formed particles, the desired topology of the particles, the relative concentration of the second emulsifier to be at the surface of the formed particles, the size of the particles, etc. For instance, ethanol can reduce the interfacial tension between water and other organic solvents, such as dichloromethane, and as such, increased levels of such a second solvent can favor the formation of smaller droplets during emulsification and similarly smaller particles upon solidification. In some embodiments, increasing the volume fraction of the second solvent in the oil phase can increase the solubility of the second emulsifier in the oil phase and as such also increase the subsequent loading of the second emulsifier in the formed particles.

In general, the oil phase will include more of the first solvent than the second solvent, i.e., the ratio of the amount of second solvent to first solvent can be less than one. In some embodiments, the ratio of the amount of the second solvent to first solvent can be from about 1:1.1 to about 1:10, or from about 1:2 to about 1:3 in some embodiments.

A system can include additional components. For instance, a system can include detectable labels (e.g., a fluorescent dye or the like). In one embodiment, a component of the system can be modified to carry a detectable label. Alternatively, a detectable label can be contained in the polymeric matrix upon solidification of the polymer. In one embodiment as described further herein, a component of the system (e.g., a polymeric first emulsifier such as polyvinyl alcohol) can be modified to carry a detectable label that can then be incorporated into the particles in conjunction with the emulsifier. A detectable label can encompass an optically detectable label, e.g., a fluorescent or phosphorescent label. For instance, isothiocyanate containing labeling agents such as fluorescein isothiocyanate (FITC), tetramethylrhodamine isothiocyanate, or rhodamine B isothiocyanate (RITC) as are readily available in the market can be utilized. Detectable labels are not limited to such materials however. Optically detectable labels can exhibit a relatively long emission lifetime and a relatively large Stokes shift. One type of fluorescent compound that has both a relatively long emission lifetime and relatively large Stokes shift are lanthanide chelates, such as chelates of samarium (Sm(III)), dysprosium (Dy(III)), europium (Eu(III)), and terbium (Tb(III)). Another type of fluorescent compound that has both a relatively long emission lifetime and relatively large Stokes shift are transition metal chelates, such as chelates of ruthenium (Ru(II)), osmium (Os(III)), and rhenium (Re(I)). These, as well as other detectable labels as are known in the art, are encompassed herein,

Other components that can provide a desired functionality to either the formation system or the formed particles can be included. By way of example, biologically active agents can be incorporated in a system that, upon particle formation, can be contained in the particles in a drug delivery application. Exemplary biologically active materials as may be incorporated in a particle can include, without limitation, proteins, carbohydrates, lipids, glycosides, indoles, peptides, polyphenols, nucleic acids, glycoproteins, glycosaminoglycans, lipoproteins, and the like.

Once an emulsion is formed including the aqueous phase that carries the first emulsifier and the dispersed organic phase that carries the polymer, the first and second solvents, and the second emulsifier, the solvents can be removed by, e.g., evaporation and/or solvent extraction. The conditions of solvent removal (reduced pressure, temperature, etc.) can encourage initial loss of the second solvent from the droplets via leaching, diffusion, etc. and, thereby, encourage movement of the second emulsifier to the water/oil interface of the system. The system can encourage a slower rate of loss of the first solvent from the droplets such that solidification occurs after the second emulsifier has moved toward the oil/water interface. Upon loss of the first solvent (which preferentially solubilizes the polymer) from the organic phase, the polymer can condense and the polymer particles can solidify. The particles can include an amount of each of the first and second emulsifiers at the surface.

Particles of any suitable size can be formed, with preferred size generally depending upon the desired application of the particles. For instance, when forming particles for use as a drug depot for in vivo delivery, limiting particle size to a range greater than about 5 microns can inhibit phagocytic clearance, and produce drug release profiles with a relatively long duration. However, smaller particles can likewise be formed.

The present disclosure may be better understood with reference to the Examples set forth below.

### Materials and Methods

75:25 poly (D,L-lactide-co-glycolide) (PLG) with a lauryl ester end group and an inherent viscosity of 0.79 dL/g was purchased from Evonik. Dichloromethane (DCM), resveratrol, poly (vinyl alcohol) (Mw 13,000-23,000, 87-89% hydrolyzed) (PVA), and fluorescein isothiocyanate (FITC) were purchased from Sigma. 200 proof ethanol (EtOH) was purchased from Decon Laboratories. Dimethyl sulfoxide (DMSO) was purchased from Fisher. Ultrapure water was obtained from a Thermo Scientific Barnstead^{TM} Nanopure^{TM} system. 3T3-L1 mouse embryonic fibroblasts (CL-173) were purchased from ATCC. Dulbecco's Modified Eagle's Medium (DMEM) with 4.5 g/L glucose and L-glutamine was purchased from Corning® cellgro®. Trypsin with 0.25% EDTA, Fetal Bovine Serum (FBS) and Pen/Strep were purchased from Fisher. Super Calf Serum was purchased from GemCell™. Insulin (bovine), dexamethasone, and methylisobutylxanthine (IBMX) were purchased from Sigma.

Scanning Electron Microscopy (SEM) was carried out by attachment of carbon adhesive tape to aluminum SEM stubs and particles were spread onto the stubs. Compressed air was applied briefly to the particles to create a monolayer on the carbon tape. Particles were sputtered with gold 3 times for 60 seconds in a Denton Desk II Vacuum sputter coater. Images were taken using a TESCAN Vega3 Scanning Electron Microscope at 10 kV.

Light microscopy images were taken on an EVOS FL microscope at 20×. Particles were prepared in ultrapure water and suspended at a concentration of 0.25 mg/m L. 400 μL of these suspensions was added to a 48-well plate and allowed to settle prior to image acquisition.

Particle size distribution was determined by analyzing light microscopy images with ImageJ software. Briefly, three representative images were acquired and converted to binary. The ImageJ Particle Analysis plugin-in was used to measure particle diameter. The size data was then plotted as a histogram with a 5 μm bin-width. The mean particle diameter and the coefficient of variation (CV %) are reported. CV % is the standard deviation divided by the mean.

Particle mass yield was calculated according to equation 1.

$$Mass Yield ( \% ) = ( \frac{{M}_{P T}}{{M}_{PLG} + {M}_{R E}} ) * 100 \qquad ( 1 )$$

where M_{PT} is the mass of particles recovered from the emulsion, M_{PLG} is the mass of PLG added to the emulsion, and M_{RE} is the mass of resveratrol added to the emulsion.

Resveratrol (or other stilbene) loading was determined by dissolving resveratrol particles in DMSO and measuring absorbance at 330 nm using a Spectramax® 190 UV-Vis spectrophotometer. A 10-point standard curve was prepared by dissolving 1 mg of empty particles and a known mass of resveratrol in DMSO. Resveratrol concentration was determined by comparing the unknown samples to the standard curve. Resveratrol loading was calculated by equation 2.

$$Loading ( \frac{μ g}{m g} ) = \frac{{M}_{R}}{{M}_{P}} \qquad ( 2 )$$

where M_{P} is the mass of particles dissolved in DMSO and M_{R} is the mass of resveratrol measured in those particles.

3T3-L1 fibroblasts (ATCC), were differentiated into adipocytes following ATCC's protocol, with slight modifications. Briefly, 3T3-L1 cells were seeded in 6 well plates at 80,000 cells/well and cultured in DMEM supplemented with 10% SuperCalf Serum and 1% Pen/Strep. 48 hours after confluence was reached, media was exchanged with “differentiation media” consisting of DMEM supplemented with 10% FBS, 10 μg/mL bovine insulin, 0.25 μM dexamethasone, and 0.5 mM IBMX. 48 hours after differentiation media was added, media was exchanged for DMEM supplemented with 10% FBS and 10 μg/mL bovine insulin. After another 48 hours, media was exchanged for DMEM supplemented with 10% FBS. Cells were then cultured until treatment with particles, which occurred 9 days after exposure to the differentiation media.

Blank PLG particles (300 μg/mL), resveratrol loaded PLG particles (300 μg/mL), or free resveratrol (60 μM) was added to 3T3-L1 adipocytes 9 days after exposure to the differentiation media. 48 hours later, cells were washed, fixed in 10% formalin, and stained with Oil Red O as follows. Cells were washed with ultrapure water and then washed with 60% isopropanol for 2 minutes. Cells were then incubated with Oil Red O for 10 minutes at room temperature. Cells were then washed four times with ultrapure water. Images were taken on a Nikon Eclipse Ci microscope using the 4× objective. Oil Red O was extracted from the wells by incubating the cells in 100% isopropanol for 10 minutes at room temperature. Absorbance of extracted Oil Red O was measured at 500 nm using a spectrophotometer.

To form FITC-labeled PVA, PVA was dissolved in DMSO at 50 mg/mL. FITC and dry KOH were added to the solution at concentrations of 3.5 mg/mL and 0.5 mg/mL, respectively. This solution was stirred at room temperature for 5 hours. The solution was then dialyzed using dialysis tubing (3.5 kDa MWCO) in 3 L of ultrapure water for 3 days, replacing the water twice daily. The FITC labeled PVA (FITC-PVA) was collected, frozen, and lyophilized. This FITC-PVA was used to fabricate PLG particles.

Confocal Microscopy—Particles were suspended in a 50:50 mixture of ultrapure water and anti-fade mounting media, pipetted on a slide, mounted with a coverslip, and dried overnight. Images were acquired on a Zeiss 700 confocal microscope using the 488 nm laser. Particles were analyzed for fluorescence intensity using ImageJ as follows. A line was drawn through each particle and the maximum intensity of FITC on the surface was recorded using the plot profile function. Data is mean±standard deviation of the measured surface pixel intensity.

Light Microscopy—Particles were suspended in ultrapure water and analyzed with a BD FACS Aria flow cytometer for FITC fluorescent intensity. Approximately 600 particles were analyzed for each condition. Data was analyzed using FlowJo® (Tree Star, Inc.).

Statistical analysis was carried out using GraphPad Prism. Where appropriate, an unpaired t-test or one-way ANOVA followed by Tukey's multiple comparison test were carried out to compare differences between means. Error bars represent the standard deviation of the means. Linear trends were determined with a Pearson's correlation test. Specific details regarding statistical analyses carried out for each data set are described in the figure legend.

### EXAMPLE 1

To prepare the PLG particles, PLG was dissolved in DCM at 6% (weight/weight, hereafter labeled as 6%) and resveratrol was dissolved in 100% ethanol at 40 mg/m L. The organic phase, which consisted of varying volume ratios of ethanol and DCM, was added to an aqueous solution of PVA at 1 or 2% (weight/volume, hereafter labeled as 1 or 2%) in a 1:7 volume ratio and homogenized using a Kinematica® PT3100D homogenizer. The emulsion was then added to an aqueous solution of PVA and stirred for 5 hours, allowing the DCM and ethanol to evaporate and the particles to harden. The volume of PVA solution in the solvent evaporation beaker was 5 times greater than the emulsion mixture. The particles were then passed through a 40 μm filter, collected via centrifugation, and washed 4 times in ultrapure water. Particles were frozen at −20° C. and lyophilized overnight. Particles were stored protected from light, under vacuum, at room temperature.

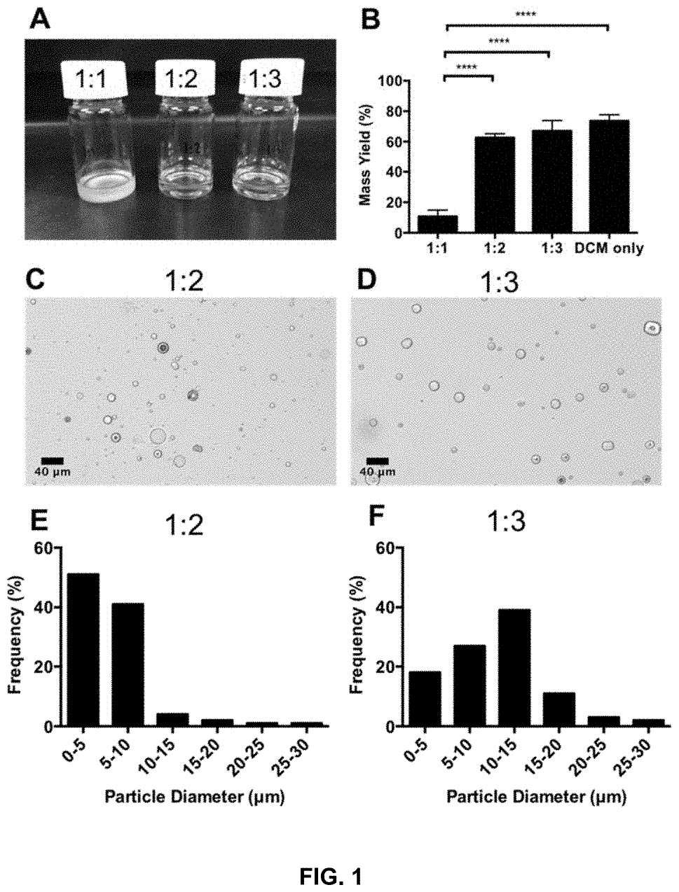

Ethanol's effect on particle size and mass yield in the absence of resveratrol was determined. 6% PLG was poorly soluble in a 1:1 mixture of ethanol and DCM (FIG. 1 at A) and few particles formed during the emulsion process, with a mass yield of 11±4% (FIG. 1 at B). In contrast, 6% PLG was soluble in a 1:2 or a 1:3 mixture of ethanol and DCM (FIG. 1 at A), and particle mass yield was similar between the two conditions, 63±3% for 1:2 versus 67±7% for 1:3 (FIG. 1 at B). Furthermore, both conditions showed similar mass yield compared to DCM alone (FIG. 1 at B). Representative light microscopy images of the 1:2 and 1:3 formulations are shown in FIG. 1 at C and D. It was found that 1:2 particles were smaller than the 1:3 particles with an average diameter of 5.7 μm versus 10.5 μm (FIG. 1 at E and F). In addition, the 1:3 particles had a similar size distribution to those made with DCM alone (data not shown). Thus, a 1:3 volume ratio of ethanol and DCM as the oil phase was used subsequently.

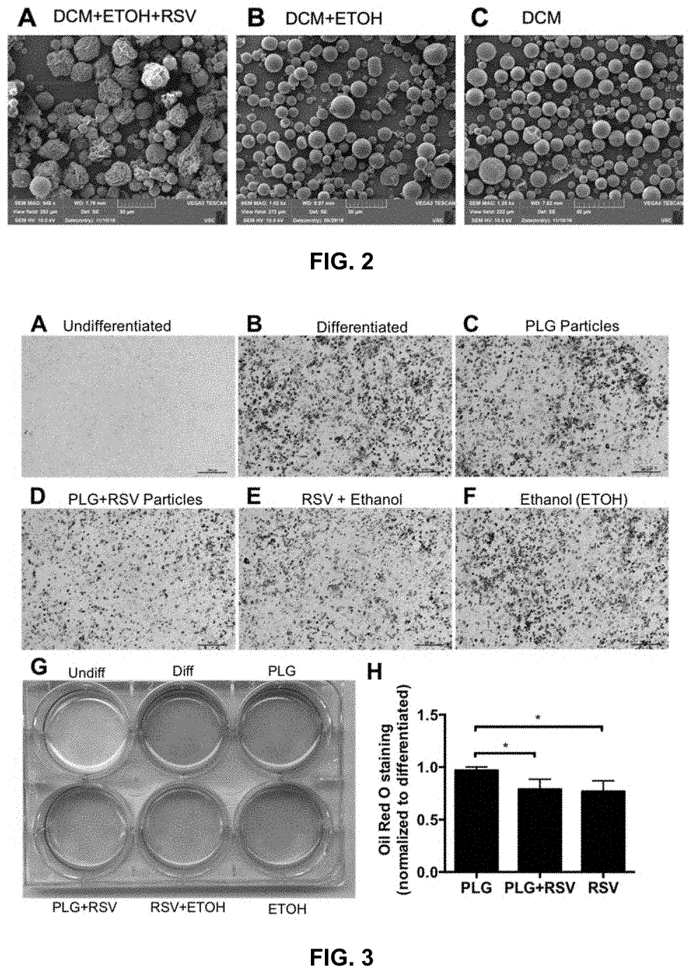

Resveratrol loaded PLG particles (from here on referred to as resveratrol particles) were formed. A 40 mg/mL solution of resveratrol in ethanol was miscible with an 8% solution of PLG in DCM when mixed at a 1:3 ratio and no precipitates formed (data not shown). This mixture was utilized for the oil phase of the emulsion, which thus contained resveratrol at 10 mg/mL and 6% PLG. This oil phase yielded particles that were irregularly shaped with ruffled surfaces (FIG. 2 at A) compared to particles made with an oil phase that consisted of only DCM and 6% PLG (FIG. 2 at C). Particles made using a 1:3 mixture of ethanol and DCM that lacked resveratrol were also smooth and spherical (FIG. 2 at B), indicating resveratrol was responsible for the altered particle morphology. Loading was 65±5 μg/mg for the resveratrol particles and mass yield was 40±5%, which was lower than the mass yield for the other two conditions, which is depicted in FIG. 1 at B.

Bioactivity of the resveratrol following the fabrication process was determined. Specifically, the ability of the resveratrol particles to induce lipolysis in differentiated 3T3-L1 adipocytes was determined by measuring lipid content with an Oil Red O assay. 3T3-L1 adipocytes are differentiated from 3T3-L1 fibroblasts and extensively used to model human adipocytes. Resveratrol promotes lipolysis in these cells, which decreases intracellular lipid content. Lipid content was visualized with Oil Red O, a lipophilic dye, (FIG. 3 at A-F) and lipid content was quantified by extracting the dye (FIG. 3 at G) and measuring the solution's absorbance (FIG. 3 at H).

The undifferentiated fibroblasts do not stain strongly with Oil Red O due to a low level of intracellular lipid (FIG. 3 at A), but 3T3-L1 adipocytes exhibit extensive staining throughout the monolayer (FIG. 3 at B). It was found that treatment of adipocytes with 300 μg/mL of resveratrol particles decreased Oil Red O staining to a similar level of cells treated with 60 μM resveratrol (FIG. 3 at D versus E and H). As controls, adipocytes were treated with particles that did not contain resveratrol (FIG. 3 at C) and ethanol, which is a vehicle control for the resveratrol treatment (FIG. 3 at F). These treatments did not decrease Oil Red O signal. Taken together, these data indicate that the resveratrol remained bioactive following its incorporation into PLG particles.

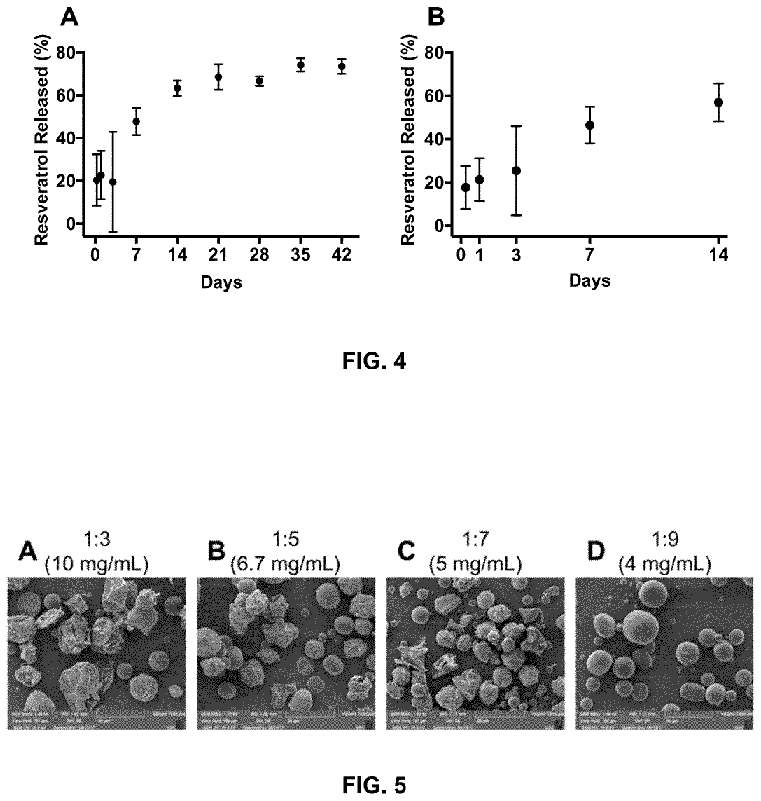

The release kinetics of resveratrol from the particles in vitro was examined. Particles were mixed in water for 6 weeks (FIG. 4 at A). Release at early time points (0.25, 1, and 3 days) was highly variable with coefficients of variation in excess of 80% at day 3. The first 14 days of the release assay was repeated once more, and variability in release was still observed (FIG. 4 at B).

Variation in the concentration of PVA in the emulsion and solvent evaporation steps was examined with regard to particle surface morphology. It was found that doubling the PVA concentration in the emulsion step (from 1 to 2%) and increasing PVA in the solvent evaporation step (from 0 to 1%) did not change particle morphology (FIG. 5 at A). The concentration of PVA in the emulsion step was increased to 5%, and the particles still exhibited irregular morphology (data not shown).

The concentration of resveratrol in the oil phase was then incrementally decreased from 10 mg/mL to 4 mg/mL by changing the volume ratio of the ethanol solution (containing 40 mg/mL resveratrol) and DCM from 1:3 to 1:9. A stark change in morphology was observed when the resveratrol concentration decreased from 5 mg/mL to 4 mg/mL, with the latter yielding spherical particles with smooth surfaces (FIG. 5).

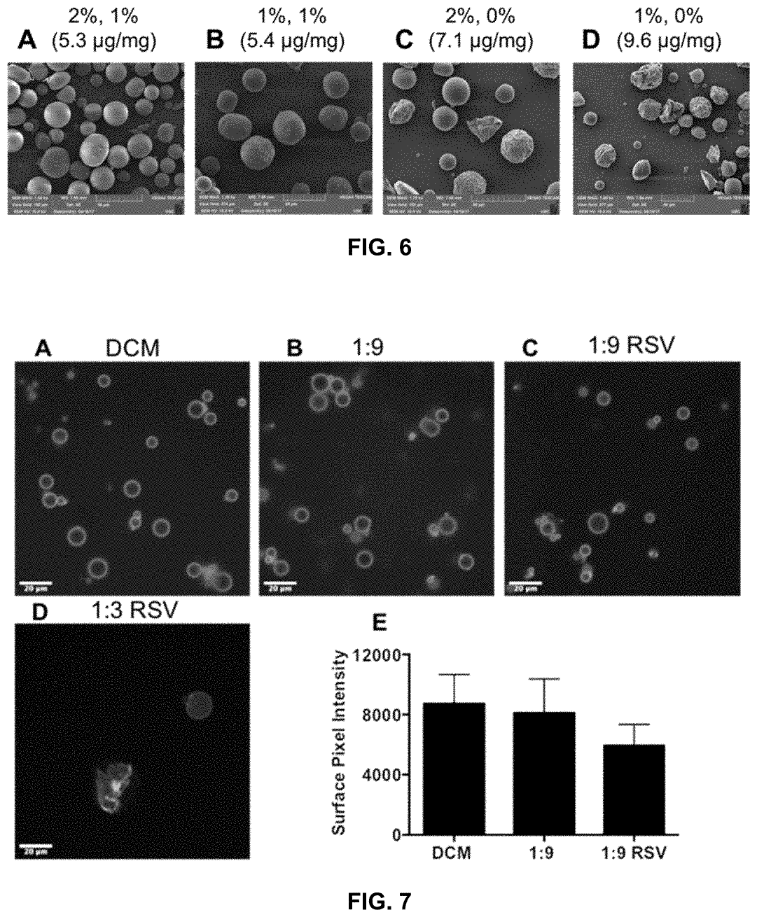

The impact on morphology of PVA concentration in both the emulsion and solvent evaporation steps was examined. It was found that a PVA concentration of 2% in the emulsion step and 1% in the solvent evaporation at 4 mg/mL resveratrol could form particles with a smooth, spherical morphology. Decreasing PVA in these cases led to irregular morphology (FIG. 6). Resveratrol loading in the particles nearly doubled (from 5.3 to 9.6 μg/mg) as PVA concentration was decreased from 2% to 1% in the emulsion step and from 1% to 0% in the evaporation step.

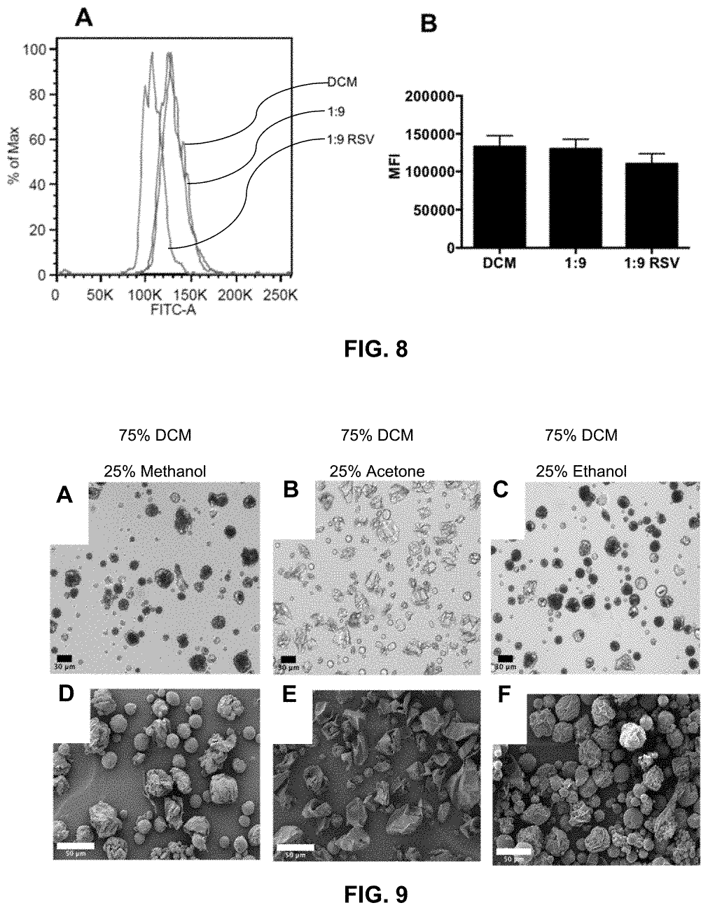

PVA content and distribution change with resveratrol loading was determined by utilizing FITC-labeled PVA (FITC-PVA). Confocal microscopy of spherical particles indicated that FITC-PVA was present on the surface of the particle and evenly distributed (FIG. 7 at A-C). This was the case whether or not resveratrol was incorporated into the particle. Particles made with a high resveratrol loading (65 μg/mg) and exhibiting a ruffled surface morphology exhibited an altered FITC signal that suggested PVA was not evenly distributed over the particle's surface (FIG. 7 at D). Image analysis revealed that the FITC signal at the surface of the spherical particles was decreased when resveratrol was encapsulated into the particle (FIG. 7 at E). To confirm the confocal data, flow cytometry of the FITC-PVA particles was conducted (FIG. 8). As shown, the FITC signal was decreased on resveratrol particles. Taken together, the data suggest that resveratrol incorporation into the particle decreased the amount of PVA that associated with the particle's surface.

### EXAMPLE 2

PLG particles were formed by dissolving PLG in DCM at 6%. Resveratrol was dissolved in either ethanol, methanol, or acetone. The organic phase was formed by addition of the resveratrol solution to the PLG/DCM solution in a 1:3 volume ratio to provide the resveratrol in the organic phase at 10 mg/m L. The organic phase was added to an aqueous solution of PVA at 1% and homogenized. The emulsion was then added to an aqueous solution of PVA and stirred for 5 hours, allowing the solvents to evaporate and the particles to harden.

FIG. 9 presents light microscopy and SEM images of the resveratrol-loaded particles formed with the different solvents including volume percentages of 25% methanol/75%% DCM at A (light) and D (SEM); 25% acetone/75%/ DCM at B (light) and E (SEM); and 25% ethanol/75% DCM at C (light) and F (SEM). All particles had a rough surface topology. Scale bars indicate 50 micrometers.

### EXAMPLE 3

Polycaprolactone (PCL) and polylactide (PLA) particles were formed by dissolving the polymer (either PCL or PLA) in DCM at 6%. Resveratrol was dissolved in ethanol and the organic phase was formed by addition of the resveratrol solution to the polymer solution in a 1:3 volume ratio to provide the resveratrol in the organic phase at 10 mg/mL. The organic phase was added to an aqueous solution of PVA at 1% and homogenized. The emulsion was then added to an aqueous solution of PVA and stirred for 5 hours, allowing the solvents to evaporate and the particles to harden. As comparison, particles were formed without resveratrol.

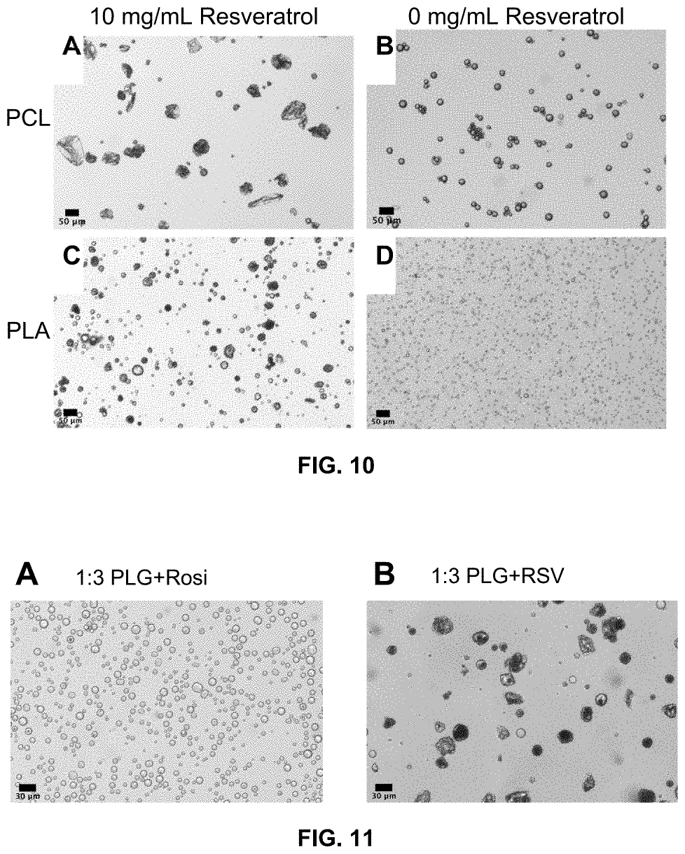

FIG. 10 presents light microscopy images of the particles made with 6% (w/w) PCL at A and B including either 10 mg/mL resveratrol (A) or no resveratrol (B) and presents light microscopy images of the particles made with 6% (w/w) PLA at C and D including either 10 mg/mL resveratrol (C) or no resveratrol (D) Scale bars indicate 50 micrometers. As can be seen, the blank particles were small and round, while the resveratrol-loaded particles were larger with a rough surface.

### EXAMPLE 4

PLG particles were formed by dissolving PLG in DCM at 6%. Either resveratrol or rosiglitazone was dissolved in ethanol. The organic phase was formed by addition of the ethanol solution to the PLG/DCM solution in a 1:3 volume ratio to provide either the resveratrol in the organic phase at 10 mg/mL or the rosiglitazone in the organic phase at 5 mg/m L. The organic phase was added to an aqueous solution of PVA at 1% and homogenized. The emulsion was then added to an aqueous solution of PVA and stirred for 5 hours, allowing the solvents to evaporate and the particles to harden.

FIG. 11 presents light microscopy images of the rosiglitazone-loaded particles at A and the resveratrol-loaded particles at B. Scale bars indicate 50 micrometers. As can be seen, the rosiglitazone-loaded particles were small and smooth, while the resveratrol-loaded particles were larger with a rough surface.

### EXAMPLE 5

Resveratrol loaded PLG particles were formed as described above. The aqueous phase was 1% (w/v) PVA. The oil phase included 6% (w/w) PLG, resveratrol, DCM and ethanol. The volume percent of ethanol and the concentration of resveratrol were varied to include ethanol at a volume percent of either 25% or 10% and varying the concentration of resveratrol from 0 mg/mL to 10 mg/m L.

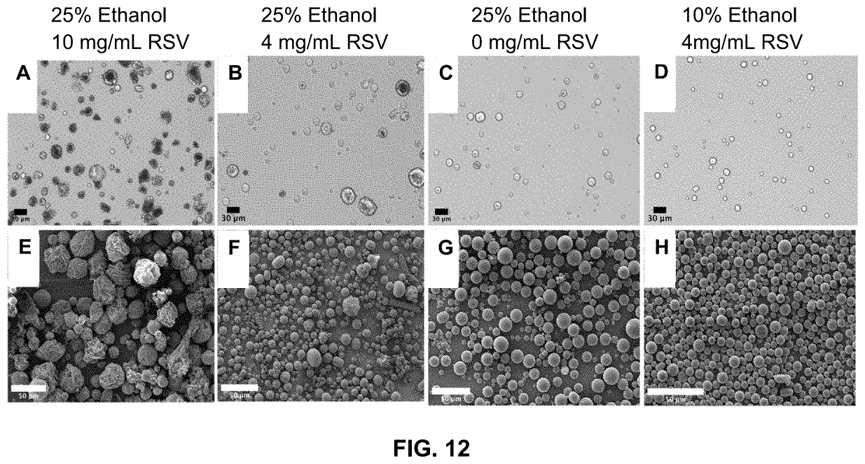

FIG. 12 presents light microscopy and SEM images of particles produced. The volume percent of ethanol and concentration of resveratrol are indicated above each column including 25% ethanol, 10 mg/mL resveratrol (A—light; E—SEM), 25% ethanol, 4 mg/mL resveratrol (B—light; F—SEM), 25% ethanol, no resveratrol (C—light; G; SEM), 10% ethanol, 4 mg/mL resveratrol (D—light; H—SEM).

EXAMPLE 6

PLG particles were formed to encapsulate several different trans-stilbenes including trans-stilbene, pinosylvin, resveratrol, and piceatannol. The particles were formed using an oil phase including 6% (w/w) PLG, 25% ethanol, 75% dichloromethane, and 10 mg/mL of the trans-stilbene of choice. The aqueous phase was 1% (w/v) PVA. The particles were formed according to an emulsion method as described previously.

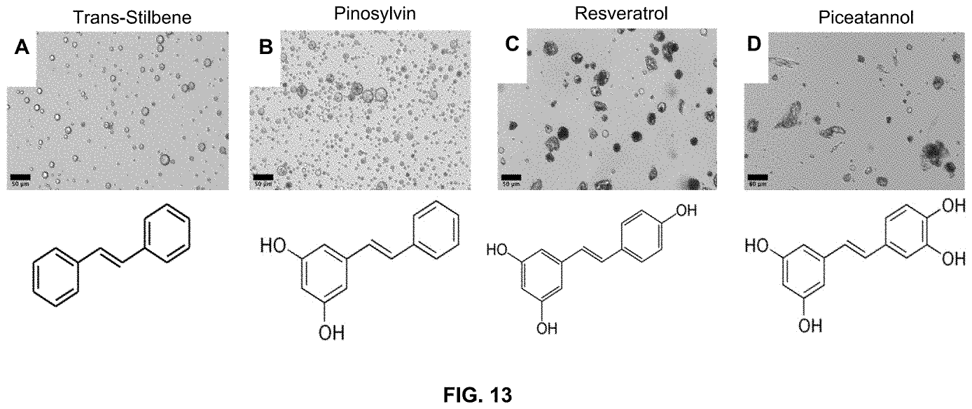

FIG. 13 presents light microscopy images of each of the products and the structure of the trans-stilbene encapsulated in the imaged particles. As indicated, the product particles varied with the different trans-stilbenes.

While certain embodiments of the disclosed subject matter have been described using specific terms, such description is for illustrative purposes only, and it is to be understood that changes and variations may be made without departing from the spirit or scope of the subject matter.

What is claimed is:

1. A method for forming a polymeric particle comprising:
combining an aqueous phase with an organic phase to form an emulsion including droplets of the organic phase dispersed in the aqueous phase, the aqueous phase comprising a first emulsifier, the organic phase comprising a second emulsifier, a first solvent, a second solvent, and a dissolved polymer, the polymer comprising a polyester homo- or copolymer, the second emulsifier comprising a small molecule amphiphilic compound; wherein
the second solvent is miscible in both water and the first solvent, the second emulsifier is more soluble in the second solvent than in the first solvent, and the polymer is more soluble in the first solvent than in the second solvent, the method further comprising:
removing at least a portion of the first solvent from the organic phase to solidify the polymer and form polymeric particles; and
modifying a concentration of the second emulsifier in the organic phase and thereby modifying the topology of the polymeric particles.

2. The method according to claim 1, the first emulsifier comprising polyvinyl alcohol, hydroxyethyl cellulose, carboxymethyl cellulose, methyl cellulose, gelatin, alkylarylsulfonates, alkylsulphates, fatty acid salts of alkali metals, polyethylene glycol, poly(ethylene-alt-maleic acid), didodecyldimethylammonium bromide, or any mixture thereof.

3. The method according to claim 1, wherein the second emulsifier has a number average molecular weight of about 300 g/mol or less.

4. The method according to claim 1, wherein the second emulsifier comprises a small molecule phenolic amphiphilic compound.

5. The method according to claim 4, wherein the second emulsifier comprises the following structure: 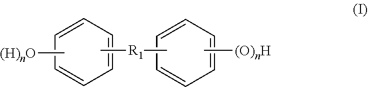
in which R₁ is alkenyl, C(O)CH═CH, or a hydroxy pyranone fused to one of the phenyl moieties to form a flavone, and each n is independently 0-3.

6. The method according to claim 1, wherein the second emulsifier comprises resveratrol (3,5,4′-trihydroxy-trans-stilbene), piceatannol (3,5,3′, 4′-tetrahydroxy-trans-stilbene), pinosylvin (trans-3,5-dihydroxystilbene), trans-stilbene, or trans-4-hydroxystilbene.

7. The method according to claim 1, the method comprising modifying the concentration of the second emulsifier in the organic phase to a concentration of about 4 mg/mL or more of the organic phase, the topology of the polymeric particles thereby being modified to exhibit a rough surface.

8. The method according to claim 1, the polymer comprising a biocompatible and biodegradable polyester.

9. The method according to claim 1, the first solvent comprising toluene, xylene, dichloromethane, chloroform, trichloroethylene, tetrachloroethylene, tetrachloroethane, chlorobenzene, dichlorobenzene, ethyl acetate, butyl acetate, ethyl formate, methylethyl ketone, or any mixture thereof.

10. The method according to claim 1, the second solvent comprising methanol, ethanol, propanol, acetone, tetrahydrofuran, or any mixture thereof.

11. The method according to claim 1, the organic phase further comprising a biologically active agent and/or a detectable label.

12. A method for forming a polymeric particle comprising:
combining an aqueous phase with an organic phase to form an emulsion including droplets of the organic phase dispersed in the aqueous phase, the aqueous phase comprising a first emulsifier, the organic phase comprising a second emulsifier, a first solvent, a second solvent, and a dissolved polymer, the polymer comprising a polyester homo- or copolymer, the second emulsifier comprising a biologically active amphiphilic phenolic compound having a number average molecular weight of about 900 g/mol or less; wherein
the second solvent is miscible in both water and the first solvent, the second emulsifier is more soluble in the second solvent than in the first solvent, and the polymer is more soluble in the first solvent than in the second solvent, the method further comprising:
removing at least a portion of the first solvent from the organic phase to solidify the polymer and form polymeric particles; and
modifying a concentration of the second emulsifier in the organic phase and thereby modifying the topology of the polymeric particles.

13. The method according to claim 12, the first emulsifier comprising polyvinyl alcohol, hydroxyethyl cellulose, carboxymethyl cellulose, methyl cellulose, gelatin, or any mixture thereof.

14. The method according to claim 12, wherein the second emulsifier has the following structure: 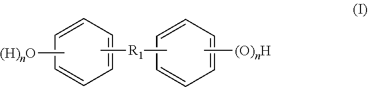
in which R₁ is alkenyl, C(O)CH═CH, or a hydroxy pyranone fused to one of the phenyl moieties to form a flavone; and each n is independently 0-3.

15. The method according to claim 14, wherein the second emulsifier comprises a polyhydroxy stilbene, a polyhydroxy chalcone, or a polyhydroxyflavone.

16. The method according to claim 12, wherein the second emulsifier comprises resveratrol (3,5,4′-trihydroxy-trans-stilbene), piceatannol (3,5,3′, 4′-tetrahydroxy-trans-stilbene), pinosylvin (trans-3,5-dihydroxystilbene), trans-stilbene, or trans-4-hydroxystilbene.

17. The method according to claim 12, the method comprising modifying the concentration of the second emulsifier in the organic phase to a concentration of about 4 mg/mL or more of the organic phase, the topology of the polymeric particles thereby being modified to exhibit a rough surface.

18. The method according to claim 12, the polymer comprising poly(ethylene oxide)-poly(propylene oxide)-poly(ethylene oxide) copolymer, poly(lactide-co-glycolide) copolymer, polycaprolactone, poly(lactic acid), poly(glycolic acid), polyethylene glycol, polysorbate, or any mixture thereof.

19. The method according to claim 12, the first solvent comprising dichloromethane, chloroform, or any mixture thereof.

20. The method according to claim 12, the second solvent comprising ethanol.

21. The method according to claim 12, the organic phase further comprising a detectable label.
